(19) United States
(12) Patent Application Publication
(10) Pub. No.: US 2006/0134131 A1
(43) Pub. Date: Jun. 22, 2006

(54) Cosmetic or pharmaceutical composition comprising a genus halopteris brown alga extract

(76) Inventors: Antoine Gedouin, Saint-Coulomb (FR); Romuald Vallee, Saint-Meloir-des-Ondes (FR); Pierre-Yves Morvan, Rennes (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/527,695

(22) PCT Filed: Sep. 10, 2003
(86) PCT No.: PCT/FR03/02691

§ 371 Date: Oct. 31, 2005

(30) Foreign Application Priority Data
Sep. 12, 2002 (FR) 02/11520

(51) Int. Cl. A61K 36/02

(52) U.S. Cl. 424/195.17

(57) ABSTRACT

The present invention concerns the use of an extract of a brown alga of the genus Halopteris or Sphacelaria in a cosmetic or pharmaceutical composition intended to limit the expansion of adipose tissues through its effect on pre-adipocyte differentiation.

The present invention concerns the use of an extract of alga in a cosmetic or pharmaceutical composition intended to limit the expansion of adipose tissues. It also concerns the said cosmetic or pharmaceutical composition intended to limit the expansion of adipose tissues.

Adipose tissues are formed from a conjunctive matrix essentially consisting of fatty cells of the hypodermic tissue, or adipocytes, which are cells which enclose a voluminous lipid vacuole in the form of triglycerides. The metabolic activity of these adipocytes comprises firstly a triglyceride synthesis step, which is referred to as lipogenesis, and secondly a triglyceride hydrolysis step referred to as lipolysis, which will release into the blood fatty acids which are used by the other cells of the organism for energy purposes.

In order to limit expansion of the adipose tissues, it is known how to use substances which can act on the adipose sites, either by stimulating the lipolysis of the intra-cellular triglycerides and the extra-cellular re-release of the fatty acids and of glycerol, or on the contrary by inhibiting the lipogenesis of new triglycerides. Extracts of plants having this type of activity have thus been able to be used in the composition of cosmetic and/or pharmaceutical products intended to trim the figure.

By way of example, the patent document FR-A-2 693 917 can be cited, which describes an aqueous extract of the brown alga Laminaria digitata, which has shown in vitro an effect of stimulating lipolysis of isolated human hypodermis adipocytes. The patent document FR-A-2 774 905 can also be cited, which describes a lipid extract of grains of Garcinia mangostana also having a lipolysis stimulation activity.

Still by way of example, the patent document FR-A-2 795 958 can be cited, which describes a lipid extract of the micro-alga Odentella aurita (with a high polyunsaturated fatty acid content) showing an effect of inhibiting the neosynthesis of fatty acids in isolated human hyperdermis adipocytes and therefore an effect of inhibiting lipogenesis, or the patent document FR-A-2 817 150, which describes a lipid extract of grains of Polygonum fogopyrum (high phytosterol content) showing a similar effect.

The present invention is more particularly concerned with brown alga of the genus Halopteris (order Sphacelaria) and, by way of example, the alga Halopteris scoparia, also referred to as Sphacelaria scoparia. The present invention concerns the use of an extract of brown alga of the genus Halopteris in a cosmetic or pharmaceutical composition intended to limit the expansion of adipose tissues. Advantageously, this alga is the alga Halopteris scoparia.

Surprisingly, it is possible to show that this extract did not have any significant effects either on the inhibition of lipogenesis nor on the stimulation of the lipolysis of triglycerides.

It has also been shown, as will be seen subsequently, that this extract has an effect on the formation of mature adipocytes, alone capable of accumulating triglycerides, from adipocyte precursors which are referred to as pre-adipocytes and which are the only ones to multiply. This transformation of the pre-adipocytes into mature adipocytes is referred to as cellular differentiation. In other words, an extract of brown alga of the genus Halopteris (or Sphacelaria) has an inhibiting effect on the differentiation of pre-adipocytes into mature adipocytes.

In general terms, cellular differentiation is usually regulated both positively and negatively by adipogenic factors present in the environment in which the cells are found, such as hormones, cytokines, growth factors, vitamins, etc. It is caused by an increase or decrease in the concentration of one or more of these adipogenic regulation factors. These regulation factors are produced either by the pre-adipocytes themselves, which is the case with a so-called autocrine regulation, or by surrounding cells, which is the case with so-called paracrine regulation, or again by cells which are further away but connected to the pre-adipocytes by blood, which is the case with a so-called endocrine regulation. In the case of adipocytes, the pre-adipocytes are cells of the fibroblastic type which, according to the surrounding factors, may differentiate either into adipocytes in the adipose tissue or into osteoblasts in the bone tissue.

From a document which appeared in Biochem. Mol. Biol. In, 1994, 32, p705-p712, entitled “Regulation of lipoprotein lipase synthesis in 3T3-L1 adipocytes by interleukin-11/adipogenesis inhibitory factor”, the authors of which were Oshumi J, Kiyadai K and Itoh Y, it is known that interleukin-11 (IL-11) has an activity of inhibiting adipocyte differentiation and therefore constitutes a negative adipogenic factor. Likewise, a document which appeared in J. Biol. Chem., 1996, 271: 615-618, entitled “Tumor necrosis factor promotes phosphorylation and binding of insulin receptor substrate 1 to phosphatidylinositol 3-kinase in 3T3-L1 adipocytes”, the authors of which were Guo D and Donner D B, showed such an effect for the alpha factor of tumour necrosis (TNFₐ).

Amongst the adipogenic factors which stimulate differentiation, insulin and insulin-like growth factor 1 (IGF-1) increase the expression of the markers of the differentiation of adipocytes such as the enzymes fatty acid synthase (FAS), glycerol-3-phosphate dehydrogenase (G₃PDH), stearol CoA desaturase (SCD), or membrane proteins such as glucose transporters (aglut-4) and fatty acid transporters (FABP) expressed on the surface of human adipocytes (see in this regard the document which appeared in J. Nutr., 1996, 126:865-870, entitled “Insulin increases lipogenic enzyme activity in human adipocytes in primary culture”, the authors of which were Moustaid N, Jones B H and Taylor J W).

Mature adipocytes produce and secrete secretion factors considered to be true specific hormones of adipocytes, such as in particular leptin and adiponectin. The latter is also Acrp 30 (Adipocyte complement-related protein of 30 Kda) according to an article by Scheren et al published in 1995 in J. Bio. Chem, 270 26746-9 or AdipoQ shown in mice according to another article by Hu et al, published in J. Bio-Chem of 1996, 271 10697-703. A similar protein was revealed in human plasma and named GBP28 (Gelatin Binding Protein of 28 Kda) or APMi (Adipose Most Abundant Gene Transcript 1) according to an article published in J. Biochem, 120, pages 803 to 812 by Nakano et al.

The results of the studies which were carried out to study the inhibiting effect on the adipocyte differentiation presented by extracts of alga of the genus Halopteris and in particular of the alga Halopteris (or Sphacelaria) scoparia are presented below.

To do this, the effect of an extract of the alga Halopteris (or Sphacelaria) scoparia on a line of pre-adipocytes 3T3-LI induced as adipocytes will be studied.

The extract of the alga Halopteris scoparia is a water-soluble extract which is obtained, in this study, by maceration of the dried or freeze-dried alga in the presence of a solvent. The solvent is a mixture of water with a co-solvent, such as for example dipropylene glycol or glycerol. The ratio between water and co-solvent is between 0 and 80%, and preferably between 30 and 50%. It should be noted that the solvent could also be only water.

The study on the effect of this extract on a line of pre-adipocytes ET3-L1 induced as adipocytes is carried out according to a protocol which is known and which is in particular described in the document which appeared in Annu. Rev. Biochem., 1995, 64: 345-373, entitled “Transcriptional regulation of gene expression during adipocyte differentiation”, the authors of which were MacDougald O A and Lane M D.

This protocol is set out briefly below. The pre-adipocyte cells of the line e=3T3-L1 were kept in culture in a Dulbelcco modified Eagle medium (DMEM) containing 2 mM of glutamine, 4.5 g/l of glucose, 0.11 g/l of sodium pyruvate, 10% of calf serum (SVD) and antibiotics (50 U penicillin and 50 μg of streptomycin per ml of medium). Their differentiation as adipocytes was brought about in the following manner: firstly, the pre-adipocyte cells were cultivated with confluence for two days. At the time which will be referred to below as D0, the culture medium was replaced by a differentiation inducing medium (DIM) composed of DMEM containing 10% foetal calf serum (FCS) with the addition of 0.25 mM of isobutyl methyl xanthine (IBMX), 0.25 μM of dexamethasone and 1.74 μM of insulin. After two days of incubation, and therefore at time D2, the induction medium was delicately withdrawn and replaced by a new culture medium containing 10% of FCS and 0.174 μM of insulin. Next the culture medium was renewed every two days.

After seven days of culture, the lipid content of the pre-adipocyte cells was assessed by colouring with Oil Red. More precisely, the pre-adipocyte cells were fixed to 3.7% formaldehyde and then incubated for ten minutes with a Oil Red colorant solution (0.5% in an isopropanol/water mixture). After 10 minutes, the cellular carpet was rinsed with water and the pre-adipocyte cells were lysed with pure isopropanol. The optical density (OD) of the medium was measured at 540 nm.

The extract of the brown alga Halopteris scoparia was tested on 3T3-L1 cells during the induction phase by means of the DIM, which therefore commenced at day D0.

In the first experimental series, this extract was added from the very start of the differentiation (at time D0) and kept present in the medium until the cells were fixed (at time D7), by renewal every two days (that is to say at time D2 and D4). The extract was tested at the following three concentrations: 0.4%, 1% and 2.5%. In the same way, use was made of a sample of 10 μM retinoic acid, which is known for inhibiting the differentiation of pre-adipocytes into adipocytes, as is in particular described in the article which appeared in Differentiation, 1990, 45:119-127, entitled “The molecular basis for inhibition of adipose conversion of 3T3-L1 cells by retinoic acid”, the authors of which were Stone R L and Berlnohr D A.

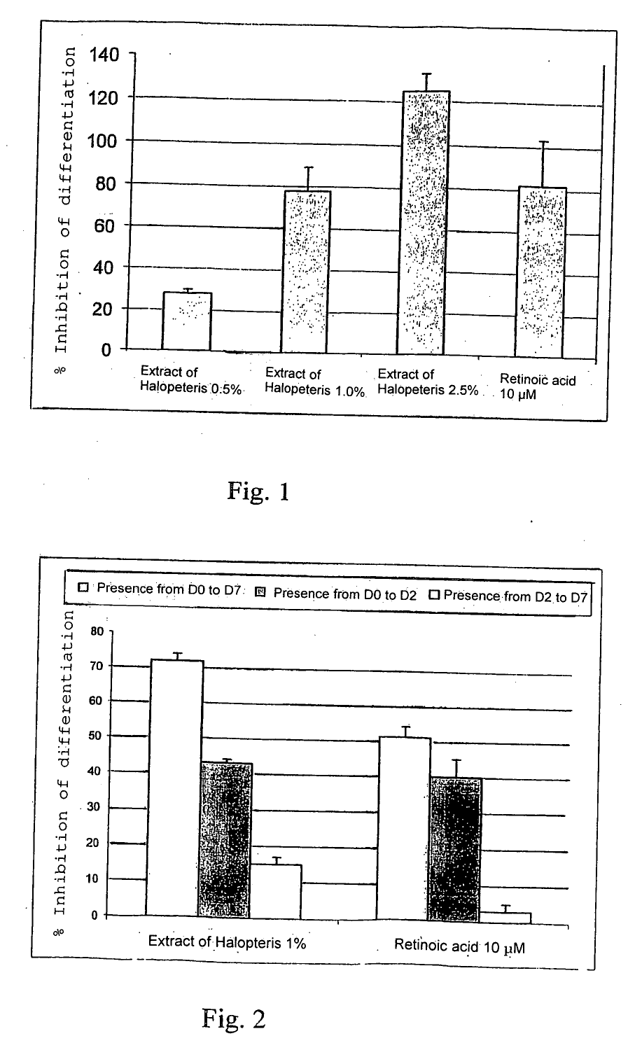

The results of these first experimental series are presented in table 1 below. Corresponding graphs are also given in FIG. 1.

TABLE 1
inhibiting effect of the extract of Halopteris
scoparia, tested at 3 non-cytotoxic concentrations,
on the differentiation of adipocytes.
Comparison with retinoic acid.
(Results obtained from a manipulation carried out in triplicate
for the extract of Halopteris, and five independent
manipulations carried out in triplicate for retinoic acid)

| | % inhibition of the |
|---|---|
| | differentiation |
| Extract of Halopteris 0.4% | 28 ± 2 |
| Extract of Halopteris 1.0% | 78 ± 11 |
| Extract of Halopteris 2.5% | 125 ± 8 |
| Retinoic Acid 10 μM | 82 ± 21 |

It will be noted that the concentrations of the extract of Halopteris are non-cytotoxic. The absence of toxicity of the extracts was assessed on the 3T3-L1 cells by the conventional method of measuring the reduction in MTT into formazan crystals (see in particular the article which appeared in J. Immunol. Methods, 1983, 65: 55-63, entitled “Rapid colorimetric assay for cellular growth and survival: application to proliferation and cytotoxicity assays”, the author of which was Mosmann T). More precisely, after incubation with the extracts, the cells were rinsed and incubated for three hours in a solution of MTT prepared at 0.5 mg/ml in a phosphate buffer (PBS). After incubation, the pre-adipocyte cells were rinsed in PBS and lysed in dimethylsulfoxide (DMSO). The D0 was measured at 540 mm.

In the second experimental series, the extract was added from the start (D0) but, unlike the first series, it was not renewed subsequently, that is to say it was not renewed after induction at time D2 nor renewed at time D4, until fixing at time D7. At the same time, cultures were treated with the extract from time D2 to time D7, with renewal at time D4.

The results of these second experimental series are presented in table 2 below. Corresponding graphs are also given in FIG. 2.

TABLE 2
kinetics of the inhibiting effect of the extract of
Halopteris scoparia, tested at 1%, on differentiation
of adipocytes. Comparison with retinoic acid.
(Results obtained for two independent manipulations
carried out in triplicate)

% inhibition of differentiation

| | Extract of Halopteris | Retinoic acid |
|---|---|---|
| | 1% | 10 μM |
| Presence from D0 to D7 | 72 ± 2 | 51 ± 3 |
| Presence from D0 to D2 | 43 ± 1 | 40 ± 5 |
| Presence from D2 to D7 | 15 ± 2 | 3 ± 2 |

As mentioned above, in the experimental series, reference cultures were produced: non-induced cells (cultivated in the culture medium with 10% SVD) and cells induced in the DIM medium, in the absence or presence of retinoic acid tested at 10 μM. The latter is in fact known for inhibiting the differentiation of pre-adipocytes into adipocytes.

It was noted that, in the absence of the differentiation inducing medium (DIM), cells in a very great majority remain pre-adipocytes. Only a few cells spontaneously produce lipid droplets visible under the microscope after colouring of the lipids by the Oil Red.

Likewise, it was found that, in the presence of the inducing medium DIM for two days, the cells to a very great extent contain a multitude of lipid droplets visible after colouring by the Oil Red.

The conclusions of these experimental series were as follows.

The extract of Halopteris scoparia, tested at 0.4%, 1% and 1.5% (v/v), inhibits the differentiation of the pre-adipocytes into adipocytes, by respectively 28%, 78% and 125%. This inhibiting effect follows a dose-dependant relationship, as can be seen in FIG. 1.

It is found that an inhibition above 100% is measured for the 2.5% extract: this shows that the presence of this extract at this non-cytotoxic concentration blocks the formulation of lipid droplets which appear spontaneously in a few cells in the absence of the inducing medium.

It is found that, under the same experimental conditions, the presence of retinoic acid at a dose of 10 μM very greatly limits the appearance of lipid droplets: the cells remain in a pre-adipocyte configuration and are not differentiated into adipocytes.

The presence of the extract of Halopteris, tested at 1% (v/v), for only the two days of induction by DIM (that is to say from time D0 to time D2) suffices to block the differentiation of the adipocytes: inhibition of 43% as against 72% when the extract is present throughout the culturing (that is to say from time D0 to time D7). When the extract is put in contact with the cells after the induction of the differentiation (that is to say from time D2 to time D7), it exerts an appreciably lower inhibiting effect: inhibition of only 15% (cf. table 2).

These results demonstrate the importance of the presence of the extract from the very start of the differentiation; a similar effect is obtained, under identical experimental conditions, with retinoic acid, tested at 10 μM (cf. FIG. 2). It should be noted that the importance of the period during which the retinoids are added to induce differentiation was widely described by Xue J C, Schwarz E J, Chawla A and Lazar M A in an article which appeared in Mol. Cell. Biol., 1996, 16:1567-1575 under the title “Distinct stages in adipogenesis revealed by retinoid inhibition of differentiation after induction of PPARgamma”.

From these experimental series, it can already be concluded that an extract of Halopteris scoparia acts as a true inhibiter of differentiation rather than as an inhibitor of lipid synthesis (lipogenesis).

In order to corroborate the results which have just been described, a new series of experiments was performed in which the effect of an extract of Halopteris scoparia on pre-adipocyte cells in the line 3T3-L1 was studied with regard to the expression of the genes coding markers normally expressed by differentiated adipocytes.

Use was therefore made as before of a culturing phase to confluence of the pre-adipocyte cells for two days, up to the time referred to as D0. There is then available a medium which serves as a reference. At time D0, the culture medium is replaced by a differentiation induction medium DIM, in the presence or not of extract of Halopteris.

At time D4 and D7 (respectively D0+4 days and D0+7 days), the total RNAs were extracted using a suitable reagent (in this case a “Tri-Reagent”) according to the protocol recommended by the supplier. The DNA was eliminated (DNA free system Ambion). The quality of the DNA was checked on agarose gel. The reverse transcription (RT) reaction of the RNAm was performed in the presence of the initiator oligo (dT) and the enzyme Superscript II (Gibco). The DNAc synthesised was quantified by fluorescence and adjusted to a concentration of 20 ng/ml. The Polymerase Chain Reaction (PCR) was performed by quantitative PCR with the “Light cycler” system from Roche Molecular Systems Inc. in accordance with the procedures recommended by the supplier. The incorporation of fluorescence in the amplified DNA is measured continuously during the PCR cycles and makes it possible to obtain the relative expression value for each marker studied.

A study was thus carried out on the expression of the transcription factor PPARg (Peroxisome proliferator activated receptor gamma) which would control the expression of the genes 422/aP2 coding for cytoplasmic proteins binding the fatty acids (FABP4), the expression of the transcription factor SREBP 1 (Sterol Response Element Binding Protein) which would control the expression of the gene coding the enzyme FAS (Fatty acid Synthase), as well as the proteins c/EBPa, c/EBPb, c/EBPg (CAAT-enhancer-binding protein alpha, beta and gamma). The factor c/EBPa controls the expression of the genes of the type 1 stearoyl CoA desaturase (SCD1) and of the glucose receptor GLUT-4. The expression of the fatty acid transporter (fatty acid translocase or FAT) was also studied.

The results obtained on the three media (reference medium, medium in the presence solely of the inducing medium and the medium in the presence of the inducing medium and the extract), for induction times of the two second media of 4 and 7 days are now given.

After 4 days of induction, the expression of c/EBPa increased by a factor of 10.4 compared with the non-induced reference. In the presence of the extract, it decreased by 33% compared with the adipocytes induced without extract. After 7 days of induction, the expression of c/EBPa increased by a factor of 27.7 compared with the non-induced reference. In the presence of the extract, it decreased by 37% compared with the adipocytes induced without the extract.

After 4 days of induction, the expression of SREBP1 increased by a factor of 1.9 (+88%) compared with the non-induced reference. In the presence of the extract, it decreased by 21% compared with the adipocytes induced in the absence of the extract. After 7 days of induction the expression of SREBP1 increased by a factor of 2.7 (+166%) compared with the non-induced reference. In the presence of the extract, it decreased by 31% compared with the adipocytes induced without the extract.

After 4 days of induction, the expression of the enzyme FAS was not increased significantly compared with the non-induced reference. However, in the presence of the extract, it decreased by 47% compared with the reference. After 7 days of induction, the expression of the enzyme FAS increased by a factor of 8.3 compared with the non-induced reference, and decreased by 58% compared with the adipocytes induced without extract.

After 4 days of induction, the SCD1 transcription factor expression increased by a factor of 14 compared with the non-induced reference. In the presence of the extract, it decreased by 25% compared with the adipocytes induced without extract. After 7 days of induction, the expression of SCD1 increased by a factor of 567 compared with the non-induced reference. In the presence of the extract, it then decreased by 27% compared with the adipocytes induced without extract.

After 4 days of induction, the expression of FAT increased by a factor of 1329 compared with the non-induced reference. In the presence of the extract, it decreased the expression of FAT by 44% compared with the adipocytes induced without extract. After 7 days of induction, the expression of FAT increased by a factor of 1528 compared with the non-induced reference. In the presence of the extract, this expression decreased by 56% compared with the adipocytes without the extract.

After 4 days of induction, the expression of GLUT-4 increased by a factor of 281 compared with the non-induced reference. In the presence of the extract, it decreased by 37% compared with the adipocytes induced without extract. After 7 days of induction, the expression of GLUT-4 increased by a factor of 3079 compared with the non-induced reference and decreased, in the presence of the extract, by 23% compared with the adipocytes induced without extract.

In accordance with the expected results, the induction medium of the DIM differentiation, without the presence of the extract of Halopteris, increased, compared with the reference culture medium, to a more or less greater extent, all the markers of the differentiation of the pre-adipocytes into adipocytes.

It can be seen that the results given above show that the extract of Sphacelaria scoparia modifies the expression of many differentiation markers for adipocytes. In particular it decreases the expression of the c/EBPa transcription factor compared with the adipocytes induced without the extract (by 33% after 4 days of induction, and by 37% after 7 days of induction), of the SREBP1 transcription factor (by 21% after 4 days of induction, and by 31% after 7 days of induction), of the enzyme FAS (by 47% after 4 days of induction, and by 58% after 7 days of induction), of the enzyme SCD1 (by 25% after 4 days of induction, and by 27% after 7 days of induction), of the fatty acid transporter FAT (by 44% after 4 days of induction and by 56% after 7 days of induction), of the glucose transporter GLUT-4 (by 37% after 4 days of induction, and by 23% after 7 days of induction), of the adipocyte protein binding the fatty acids FABP4 (by 18% after 4 days of induction). On the other hand, it did not show any significant effect on the PPARg factor. However, the extract modulates the expression of two genes which would be under the control of the PPARg factor: it decreases (at least temporarily) the expression of FABP4.

It also decreases the expression of the micro-protein lipase (LPL). After 7 days of induction the expression of the LPL induced by the induction medium is decreased by 66% in the presence of the extract.

It was also shown that, after 4 days of induction, the expression of COL1 decreased by 70% compared with the non-induced reference. In the presence of the extract, this expression decreased only by 46% compared with the non-induced reference, which means that it increased by 79% compared with the adipocytes induced without extract. After 7 days of induction, the expression of COL1 decreased by 80% compared with the non-induced reference. In the presence of the extract, its expression decreased only by 59% compared with the non-induced reference, which means that it increased by a factor of 2 compared with the adipocytes induced without extract.

After 4 days of induction the expression of COL4 increased by a factor of 3.1 compared with the non-induced reference. In the presence of the extract, it increased by 16% compared with the adipocytes induced without extract. After 7 days of induction, the expression of COL4 increased by a factor of 2.6 compared with the non-induced reference. In the presence of the extract, it increased by 17% compared with the adipocytes induced without extract.

Thus the addition of the inducing medium DIM reduced the expression of type 1 collagen (COL1). This collagen is most abundant in the dermis. It forms thick fibres whose main property is tensile strength. It is produced principally by fibroblasts, but also by cartilaginous cells and osteoblasts; these cells derive from the fibroblasts like the adipocytes. It makes it possible to keep the adipocytes attached to one another. During differentiation, the pre-adipocytes therefore lose their capacity to produce type I collagen. On the other hand, mature adipocytes produce type 4 collagen (COL4). This does not form fibres. It is situated in a non-organised form in the basal laminae, in particular in the dermo-epidermic basal membrane. COL4 serves as a filtration support whilst being situated intermediate between the support tissue (the dermis) and the epithelial tissue (the epidermis). It is manufactured by the epithelial cells (keratinocytes) and the endothelial cells. It is also produced by the adipocytes.

It can be seen that the extract modifies the expression of collagens: it decreases the loss of expression of type 1 collagen (COL1) normally observed during differentiation. The expression of COL1 increases compared with the adipocytes induced without extract (by 79% after 4 days of induction and 59% after 7 days of induction). Moreover, the extract does not prevent the expression of type 4 collagen (COL4), which is normally increased during the differentiation of the adipocytes. The extract increases the expression of COL4 compared with the adipocytes induced without the extract (by 16% after 4 days of induction and 17% after 7 days of induction).

Analysis of the expression of the genes of the differentiation of adipocytes by the RTPCR method therefore confirms the inhibiting effect of the extract of Halopteris scoparia on the differentiation of the pre-adipocytes into mature adipocytes. It also shows that this effect is restricted to certain specific markers of the differentiation. In addition it keeps high the level of type 1 collagen (COL1) whilst stimulating the expression of type 4 collagen (COL4). This effect is interesting having regard to the need for restructuring of dermal tissue following an attack on the adipose tissue by lipolytic agents.

It was also shown that the extract of Halopteris scoparia (at 1%) reduced the expression of adiponectin by 70% compared with the adipocytes induced in the absence of the extract.

Consequently the present invention concerns the use of an extract of a brown alga of the genus Halopteris in a cosmetic or pharmaceutical composition intended to limit the expansion of adipose tissues through its effect on pre-adipocyte differentiation. The said alga is advantageously the alga Halopteris scoparia.

Moreover, according to one advantageous embodiment, the said extract is obtained by the maceration of dried or freeze-dried algae in the presence of a solvent, such as water, or a mixture of water with a co-solvent. The said co-solvent may be dipropylene glycol or glycerol, the ratio between water and co-solvent being between 0 and 80%, advantageously between 30 and 50%.

The present invention also concerns a cosmetic or pharmaceutical composition intended to limit the expansion of adipose tissues, which is characterised in that it comprises, as an active product, at least one extract of a brown alga of the genus Halopteris which acts so as to inhibit adipocyte differentiation. The said alga is advantageously the alga Halopteris scoparia or Sphacelaria scoparia.

The said extract is advantageously obtained by the maceration of dried or freeze-dried algae in the presence of a solvent, such as water, or a mixture of water with a co-solvent. The said co-solvent may be dipropylene glycol or glycerol, the ratio between water and co-solvent being between 0 and 8%, advantageously between 30 and 50%.

According to another characteristic of the present invention, there are associated, in a cosmetic formulation for slimming purposes, an extract of alga of Halopteris, an active agent stimulating lipolysis (such as for example an inhibitor for phosphodiesterase or an agent blocking the adrenergic receptors), and/or an active agent inhibiting lipogenisis (such as for example an inhibitor of the enzyme fatty acid synthase or an agent blocking the glucose penetration receptors).

The present invention also concerns the use of a cosmetic composition as just described, for the purpose of limiting the expansion of adipose tissues by inhibiting pre-adipocyte differentiation.

1. Use of an extract of a brown alga of the genus Halopteris in a cosmetic or pharmaceutical composition intended to limit the expansion of adipose tissues by its effect on pre-adipocyte differentiation.

2. Use according to claim 1, wherein the said alga is the alga Halopteris scoparia.

3. Use according to claim 1, wherein the said extract is obtained by the maceration of dried or freeze-dried alga in the presence of a solvent, such as water, or a mixture of water with a co-solvent.

4. Use according to claim 3, wherein the said co-solvent is dipropylene glycol or glycerol.

5. Use according to claim 4, wherein the ratio between water and co-solvent is between 0 and 80%.

6. Use according to claim 5, wherein the ratio between water and co-solvent is between 30 and 50%.

7. Cosmetic or pharmaceutical composition intended to limit the expansion of adipose tissues, wherein it comprises, as an active product, at least one extract of the brown alga of the genus Halopteris which acts so as to inhibit adipocyte differentiation.

8. Cosmetic or pharmaceutical composition according to claim 7, wherein the said alga is the alga Halopteris scoparia.

9. Cosmetic or pharmaceutical composition according to claim 7, wherein the said extract is obtained by the maceration of dried or freeze-dried alga in the presence of a solvent, such as water, or a mixture of water with a solvent.

10. Cosmetic or pharmaceutical composition according to claim 9, wherein the said co-solvent is dipropylene glycol or glycerol.

11. Cosmetic or pharmaceutical composition according to claim 9, wherein the ratio between water and co-solvent is between 0 and 80%.

12. Cosmetic or pharmaceutical composition according to claim 9, wherein the ratio between water and co-solvent is between 30 and 50%.

13. Cosmetic or pharmaceutical composition according to claim 7, wherein it also comprises an active stimulating lipolysis and/or an active agent inhibiting lipogenesis.

14. Use of a cosmetic composition according to claim 7, for the purpose of limiting the expansion of adipose tissues by inhibiting pre-adipocyte differentiation.
